(19) United States
(12) Patent Application Publication
(10) Pub. No.: US 2017/0156601 A1
(43) Pub. Date: Jun. 8, 2017

(54) SUBJECT INFORMATION ACQUISITION APPARATUS AND SUBJECT INFORMATION ACQUISITION METHOD

(71) Applicant: CANON KABUSHIKI KAISHA, Tokyo (JP)

(72) Inventors: Yoshiaki Sudo, Chigasaki-shi (JP); Kazuhiko Fukutani, Kyoto-shi (JP); Takao Nakajima, Kyoto-shi (JP); Yoshitaka Baba, Tokyo (JP)

(21) Appl. No.: 15/383,632

(22) Filed: Dec. 19, 2016

Related U.S. Application Data
(63) Continuation of application No. 13/513,341, filed on Jun. 1, 2012, now abandoned.

(30) Foreign Application Priority Data
Jan. 27, 2010 (JP) 2010-015535

(51) Int. Cl. A61B 5/00; G01N 29/24

(52) U.S. Cl. CPC A61B 5/0095; G01N 29/2418; G01N 2291/02475

(57) ABSTRACT

A subject information acquisition apparatus includes: an acoustic wave detector which detects an acoustic wave which is generated from a subject by irradiating light and outputs a detection signal; an amplifier which amplifies the detection signal which is output from the acoustic wave detector; a gain control unit which changes a gain of the amplifier as time elapses, according to a gain control table, in order to correct a drop in intensity of the acoustic wave caused by attenuation of fluence inside the subject; and a signal processing unit which obtains information inside the subject based on the signal amplified by the amplifier. Measurement under a plurality of measurement conditions, where at least fluence distribution inside the subject or a position of the acoustic wave detector differs, is possible, and the gain control unit changes the gain control table according to the measurement conditions.

### RELATED APPLICATIONS

This is a divisional of application Ser. No. 13/513,341, filed Jun. 1, 2012, which is a national-phase application of PCT/JP2011/000412, filed Jan. 26, 2011, claims benefit of that application under 35 U.S.C. §120, and claims benefit under 35 U.S.C. §119 of Japanese patent application no. 2010/015535, filed Jan. 27, 2010. The entire contents of each of the mentioned prior applications are incorporated herein by reference

### TECHNICAL FIELD

The present invention relates to a subject information acquisition apparatus and a subject information acquisition method.

### BACKGROUND ART

Imaging apparatuses that use X-rays, ultrasonic waves and nuclear Magnetic Resource Imaging (MRI) are frequently used in medical fields. Research on optical imaging apparatuses which obtain information on biological tissues by propagating light irradiated from such a light source as a laser onto a subject, such as biological tissue, and detecting the propagated light, is vigorously progressing. Photoacoustic tomography (PAT) has been proposed as one optical imaging technologies (NPL1).

PAT is a technology to visualize information related to the optical property values inside a subject by irradiating pulsed light, generated from a light source, onto the subject, detecting acoustic waves generated from biological tissue which absorbed the energy of the light propagated and diffused in the subject at a plurality of locations, and analyzing these signals. Thereby the optical property distribution inside the subject, particularly the optical energy absorption density distribution, can be obtained.

According to NPL1, the initial sound pressure (P0) of the acoustic wave, generated from a light absorber in the subject due to light absorption, can be expressed as follows.

[Math. 1]

P₀=Γ·μa·φ Eq. (1)

where

Γ: Grüneisen coefficient

μa: light absorption coefficient of light absorber

φ: local fluence irradiated onto light absorber

μa·φ: optical energy absorption density distribution

According to NPL1, the initially generated sound pressure distribution (P0) of the subject can be imaged by measuring the change of the sound pressure P, which is a magnitude of the acoustic wave, at a plurality of locations, and analyzing the data. The Grüneisen coefficient is a product of a thermal expansion coefficient and a square of sound velocity, divided by the specific heat at constant pressure, and is known to become an approximate constant value if a tissue is determined. Therefore based on the relationship of Eq. (1), the product of the light absorption coefficient and the fluence distribution, that is, the optical energy absorption density distribution, can also be obtained.

### CITATION LIST

### Non-Patent Literature

NPL 1: M. Xu, L. V. Wang, “Photoacoustic imaging in biomedicine”, Review of Scientific Instruments, 77, 041101(2006)

### SUMMARY OF INVENTION

In the case of photoacoustic tomography, in order to determine the light absorption coefficient distribution in the subject based on the result of measuring the sound pressure, distribution of fluence irradiated onto the absorber, which generates acoustic waves, must be determined by some method, as Eq. (1) shows. However the light introduced into a subject (particularly biological tissue) is diffused and attenuated, so it is difficult to estimate fluence irradiated onto the absorber. Therefore conventionally only a distribution of optical energy absorption density or distribution of an initial pressure (P0), which is the distribution of optical energy absorption density multiplied by a Grimeisen coefficient, can be imaged based on the result of measuring the sound pressure of the acoustic wave. In other words, a problem is that light absorbers having the same size, shape and absorption coefficient are displayed with different contrasts, because of the influence of attenuation of fluence in the biological tissue (that is, depending on which area of the tissue the light absorber exists).

With the foregoing in view, it is an object of the present invention to provide a technology for decreasing the influence of attenuation of fluence inside the subject in photoacoustic tomography.

The present invention in its first aspect provides a subject information acquisition apparatus including: an acoustic wave detector which detects an acoustic wave which is generated from a subject by irradiating light and outputs a detection signal; an amplifier which amplifies the detection signal which is output from the acoustic wave detector; a gain control unit which changes a gain of the amplifier as time elapses, according to a gain control table which defines a time-based change of the gain, in order to correct a drop in intensity of the acoustic wave caused by attenuation of fluence inside the subject; and a signal processing unit which obtains information inside the subject based on the signal amplified by the amplifier, wherein measurement under a plurality of measurement conditions, where at least fluence distribution inside the subject or a position of the acoustic wave detector differs, is possible, and the gain control unit changes the gain control table according to the measurement conditions.

The present invention in its second aspect provides a subject information acquisition method including the steps of: detecting an acoustic wave which is generated from a subject by irradiating light, and outputting a detection signal; changing a gain of an amplifier as time elapses, upon amplifying the detection signal which has been output by the amplifier, according to a gain control table which defines the time-based change of the gain, in order to correct a drop in intensity of the acoustic wave caused by attenuation of fluence inside the subject; and obtaining information from the subject based on the signal amplified by the amplifier, wherein measurement under a plurality of measurement conditions, where at least fluence distribution inside the subject or a position of the acoustic wave detector differs, is possible, and the gain control table is changed according to the measurement condition.

According to the present invention, the influence of attenuation of fluence inside the subject can be decreased in photoacoustic tomography.

Further features of the present invention will become apparent from the following description of exemplary embodiments with reference to the attached drawings.

### BRIEF DESCRIPTION OF DRAWINGS

FIG. 1 is a diagram depicting a subject information acquisition apparatus of the first embodiment.

FIG. 2A is a graph depicting fluence distribution inside a subject.

FIG. 2B is a graph depicting fluence distribution inside a subject.

FIG. 2C is a graph depicting fluence distribution inside a subject.

FIG. 3A is a graph depicting an example of a gain control table used for TGC.

FIG. 3B is a graph depicting an example of a gain control table used for TGC.

FIG. 3C is a graph depicting an example of a gain control table used for TGC.

FIG. 4 is a diagram depicting a configuration example of an amplifier and a gain control unit.

FIG. 5 is a diagram depicting a subject information acquisition apparatus of a second embodiment.

FIG. 6 is a graph depicting a change of fluence distribution when the distance between compressing plates is changed.

FIG. 7 is a graph depicting a change of a gain control table when the distance between compressing plates is changed.

### DESCRIPTION OF EMBODIMENTS

A subject information acquisition apparatus according to the present embodiment is an imaging apparatus using photoacoustic tomography (PAT). This subject information acquisition apparatus has: a light source which irradiates light onto a light irradiation region on the subject; and an acoustic wave detector, which detects an acoustic wave generated by a light absorber in the subject absorbing the light, and outputs a detection signal. The subject information acquisition apparatus also has: an amplifier which amplifies the detection signal being output from the acoustic wave detector; a gain control unit which controls a gain of the amplifier; and a signal processing unit which obtains information (e.g. optical property distribution) from the subject based on the signal amplified by the amplifier. In the present invention, an acoustic wave includes a sonic wave, ultrasonic wave and photoacoustic wave, and refers to an elastic wave which is generated inside the subject by irradiating such light as near infrared rays (electromagnetic wave) onto the subject.

The light which entered from the light irradiation region (surface where light is irradiated) into the subject is diffused inside the subject, so fluence (number of photons) dramatically decreases as distance from the light irradiation region increases. In other words, the intensity of the acoustic wave to be generated (initial sound pressure) decreases inside the subject as it becomes distant from the surface where light is irradiated. Therefore according to the present embodiment, the gain of the amplifier is changed by the gain control unit in order to correct the drop in intensity of an acoustic wave caused by this attenuation of fluence. In concrete terms, the gain of the amplifier is increased as the area where the detection target acoustic wave is generated is more distant from the light irradiation region.

If the subject information acquisition apparatus can measure a plurality of measurement conditions where the light irradiation method and/or the position of the acoustic wave detectors are/is different, the way of changing the gain must be changed depending on the measurement conditions. This is because if the light irradiation method (e.g. position, number and size of the light irradiation region(s), fluence and frequency of irradiated light) changes, the fluence distribution inside the subject, that is, the intensity of the acoustic wave at each point inside the subject, changes, and if the position of the acoustic wave detector changes, the detection time of the acoustic wave changes. For example, if the light irradiation region is at the opposite side of the acoustic wave detector with respect to the subject, the acoustic wave that enters the acoustic wave detector increases intensity as the time elapses. Hence control to decrease the gain of the amplifier as the time elapses is required. On the other hand, if the light irradiation region is on the same side as the acoustic wave detector, the acoustic wave that enters the acoustic wave detector decreases intensity as the time elapses. Therefore control to increase the gain of the amplifer as the time elapses is required.

With the foregoing in view, the subject information acquisition apparatus of this embodiment uses a configuration where a gain control table, which the gain control unit uses for controlling gain, is changed according to the measurement conditions used for measurement. In the case of this configuration, fluence distribution, detection time or the like need not be calculated each time, so adaptive gain control can be implemented with a simple circuit configuration. The gain control table is a function or a table for defining time-based change of the gain (or a value of gain with respect to the time elapsed from the light irradiation time).

The following two configurations are available to change the gain control table. One is the gain control unit storing in memory, in advance, a plurality of gain control tables corresponding to each of the plurality of measurement conditions, so that a gain control table corresponding to the measurement condition is selected upon measurement, and is used for gain control. Since a dedicated table is provided for each measurement condition, high correction accuracy can be expected. This configuration is advantageous if variations of the measurement condition are limited. The other configuration is the gain control unit storing, in advance, a standard gain control table corresponding to a standard measurement condition, and correcting the standard gain control table based on the difference of measurement condition upon measurement and the standard measurement condition, so that the corrected table is used for gain control. The advantage of this configuration is that the cost required to create tables and memory capacity for storing the tables can be decreased. It is certainly preferable as well to combine these two configurations.

This technology to change the gain of the amplifier as the time elapses is called “TGC (Time Gain Control)”. TGC is also used for conventional ultrasonic diagnostic apparatus (apparatus for transmitting ultrasonic waves and receiving the reflected ultrasonic waves reflected by a measurement target in the subject). The purpose thereof, however, is for correcting attenuation of the ultrasonic wave signal in biological tissue, and not for correcting the drop in intensity of the acoustic wave caused by the attenuation of fluence, like the case of this embodiment. In the case of an ultrasonic diagnostic apparatus in which an ultrasonic probe both generates and detects ultrasonic waves, gain is simply increased as time elapses. In the case of the TGC of this embodiment, on the other hand, the way of changing gain is changed according to the measurement conditions. In this aspect as well, the TGC of this embodiment and a conventional TGC are different.

A variable gain amplifier is a circuit to implement the TGC of this embodiment. The variable gain amplifier is an amplifier which can change the gain using an input signal from the outside, unlike a normal amplifier. The input to control gain is normally voltage control. In other words, the TGC uses a variable gain amplifier as the amplifier for amplifying acoustic wave detection signals, and controls the input signal for changing gain of the variable gain amplifier as time elapses.

When measurement is performed using a plurality of acoustic wave detectors, or when the acoustic wave detector is constituted by a plurality of elements which convert received acoustic waves into electric signals, a same gain control table can be used for all the acoustic wave detectors, if the distance from the light irradiation region on the subject to each acoustic wave detector or each element is the same. However if the distance from the light irradiation region to the acoustic wave detector is different, like the configuration of a plurality of acoustic wave detectors or a plurality of elements are arrayed in a direction intersecting orthogonally to the light irradiation region, it is preferable to change the gain control table for each acoustic wave detector or for each element. In other words, the intensity of the detected signal of an acoustic wave detector or an element disposed closer to the light irradiation region is relatively stronger than that of the acoustic wave detector or element disposed more distant, so gain is decreased as the distance from the light irradiation region decreases. By adjusting gain for each acoustic wave detector or each element according to the distance from the light irradiation region, in addition to TGC, further improvement of correction accuracy can be expected.

The preferred embodiments of this invention will now be described in detail with reference to the drawings.

### First Embodiment

### (Configuration of Apparatus)

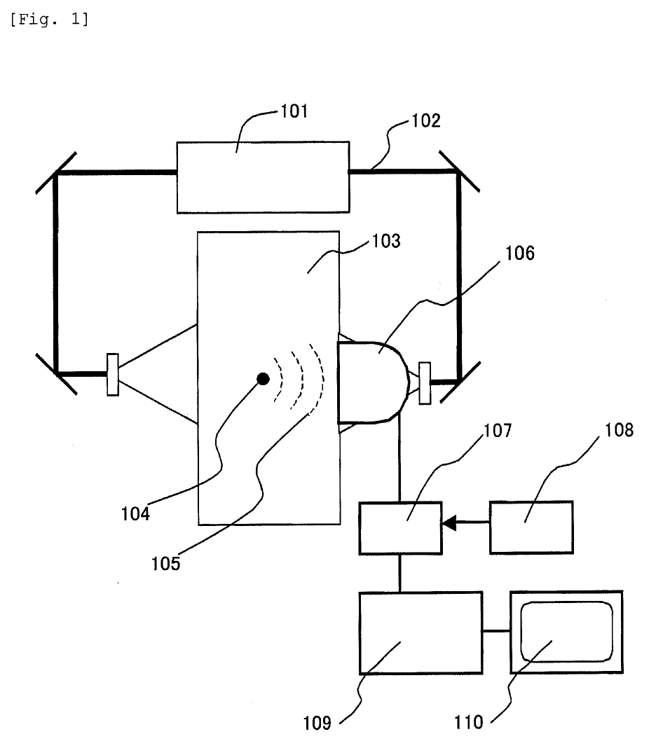

FIG. 1 is a diagram depicting a configuration of a subject information acquisition apparatus according to a first embodiment of the present invention. The first embodiment of the present invention will be described with reference to FIG. 1. The subject information acquisition apparatus to be described here is an apparatus which can image optical property distribution in biological tissue and density distribution of a substance constituting biological tissue, which is obtained from this information, for such purposes as diagnosing a malignant tumor, vascular disorder or the like, and observing the progress of chemotherapy.

The subject information acquisition apparatus is comprised of a light source 101, an acoustic wave detector (also called a “probe”) 106, an amplifier 107, a gain control unit 108, a signal processing unit 109 and a display device 110. The light source 101 is a device which emits light 102. The light 102 emitted from the light source 101 is irradiated onto a subject 103, such as biological tissue. The light source 101 of this embodiment has a configuration which allows irradiating light onto the subject 103 from both sides. In the case of the example in FIG. 1, the light 102 from a single light source 101 is split by optical devices, and is guided to both side of the subject 103, but another desirable configuration is irradiating light from a plurality of directions by simultaneously emitting a plurality of light sources which are disposed individually for each light irradiation region. If a light absorber 104 existing on the surface layer on one side of the subject 103 is detected, the light may be irradiated only onto one side. In such a case, the optical path is switched so that the light from the light source 101 is irradiated only onto one side.

If a part of the energy of the light propagating inside the subject 103 is absorbed by such a light absorber 104 as a blood vessel, an acoustic wave (ultrasonic wave in particular) 105 is generated from the light absorber 104. The acoustic wave 105 generated from the light absorber 104 is detected by an acoustic wave detector 106, which is contacted to the subject 103, and is converted into electric signals. An amplifier 107 amplifies the electric signals which are output from the acoustic wave detector 106. A signal processing unit 109 converts the electric signals amplified by the amplifier 107 into digital signals, and then performs signal processing. This signal processing is processing for generating image data using the signals converted into digital signals, and generates the optical property distribution inside biological tissue, and density distribution of the substance constituting the biological tissue obtained from this information as image data (reconstructs an image). The signal processing unit 109 is a personal computer (PC), for example. The display device 110 is a device for displaying the image data generated by the signal processing unit 109 as an image.

### (Attenuation of Fluence)

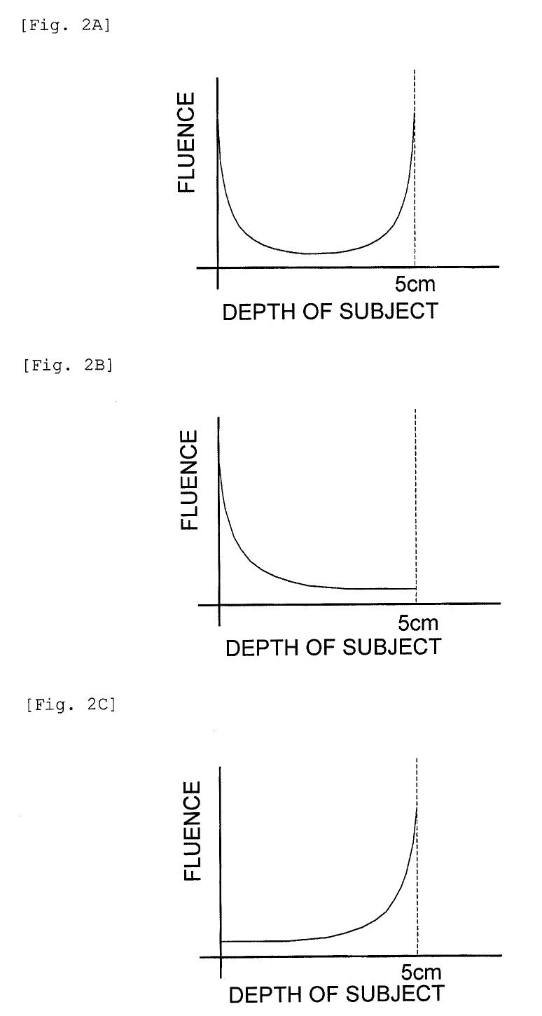

FIG. 2A is a graph depicting the fluence distribution inside the subject when the light is irradiated onto the subject 103 from both sides. The abscissa is a depth from the light irradiation surfaces on the side of the acoustic wave detector 106 (light irradiation surface on the right side in FIG. 1). FIG. 2A is an example when the distance between the irradiation surface on both sides of the subject is 5 cm. In the same manner, FIG. 2B show the fluence distribution when the light is irradiated onto the subject 103 from the right side, and FIG. 2C shows the fluence distribution when the light is irradiated onto the subject 103 from the left side. In the case of one side irradiation, in the subject 103 the fluence attenuates exponentially with respect to the distance from the irradiation surface. If the light is irradiated from both sides, as shown in FIG. 2A, the fluence is higher as the area is closer to the irradiation surfaces on both sides, and attenuates exponentially as the area is closer to the center between the irradiation surfaces. The center portion receives a contribution of the fluence on both sides. This fluence distribution can be calculated by a Monte Carlo method or finite element method, for example, based on the disposition of light irradiation regions in the subject, fluence and frequency of irradiation light, and optical coefficients (optical property values), such as the light absorption and light scattering, inside the subject. Instead of such a numerical calculation method, the fluence distribution may be calculated based on an analytic solution.

The change of fluence inside the subject, as shown in FIG. 2A to FIG. 2C, influences the initial sound pressure of the acoustic wave to be generated. In other words, the initial sound pressure of the acoustic wave to be generated is in proportion to the light quantity. If the initial sound pressure is high, the intensity of the acoustic wave to be detected by the acoustic wave detector increases in proportion. In other words, the intensity of the acoustic wave to be detected is in proportion to the fluence inside the subject.

The acoustic wave generated from the light absorber attenuates while propagating inside the subject 103. It is preferable to correct attenuation considering this attenuation of the acoustic wave as well for accurate measurement, although the attenuation correction of the acoustic wave is not mentioned in this embodiment.

### (Gain Control)

In order to correct the above mentioned intensity change of an acoustic wave due to attenuation of fluence inside the subject, the gain of the amplifier 107 for amplifying the detection signal of detected acoustic waves is controlled by the gain control unit 108, according to the present embodiment. The acoustic waves generated from an area closer to the acoustic wave detector 106 reach the acoustic wave detector first, and the acoustic waves generated from a more distance area reach the acoustic wave detector later. By continuously changing over time from a gain for amplifying a detection signal from an area closer to the acoustic wave detector 106 to a gain for amplifying a detection signal from an area more distant, the attenuation of fluence can be corrected.

The gain control unit 108 stores, in advance, a gain control table for defining the time-based change of the gain provided to the amplifier 107, and changes the gain of the amplifier 107 according to this gain control table. The gain control table can be determined from the fluence distribution inside the subject and the position of the acoustic wave detector 106. In other words, if the fluence distribution inside the subject is known, the intensity (initial sound pressure) of the acoustic wave generated in each area inside the subject can be determined, and if the position of the acoustic wave detector 106 is determined, the time when an acoustic wave arrives from each area inside the subject can be determined. By deciding the value of the gain so as to decrease the dispersion of the initial sound pressure of each area, and arranging these values in the sequence of the arrival time of a respective acoustic wave, the gain control table is obtained. In reality, it is preferable to determine the inclination of the curve and the absolute value of the gain considering the value of initial sound pressure of each area, sensitivity of the acoustic wave detector 106, noise of the amplifier 107 and controllability, among other factors.

As FIG. 2A to FIG. 2C show, if conditions of the light irradiation onto the subject are changed, the fluence distribution inside the subject changes. Therefore according to this embodiment, a plurality of gain control tables corresponding to each measurement condition are prepared in advance, and the gain control unit 108 switches the gain control tables according to the condition upon measurement.

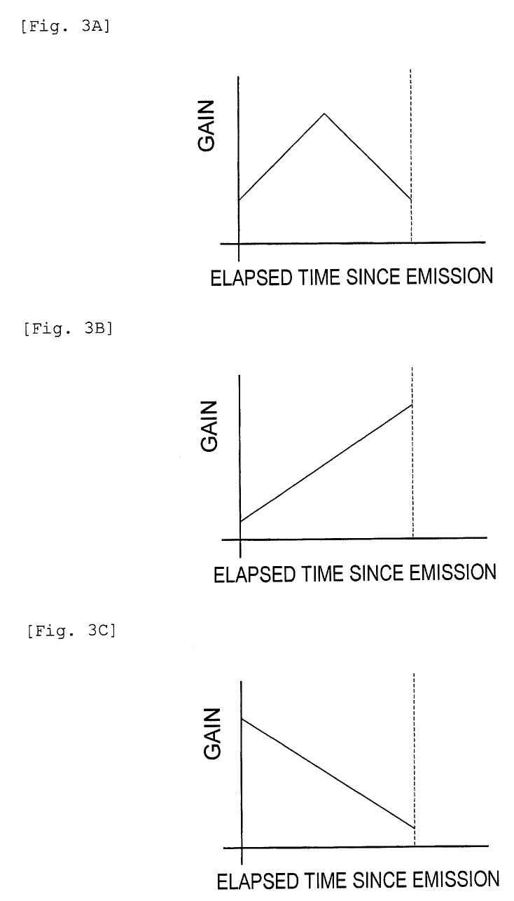

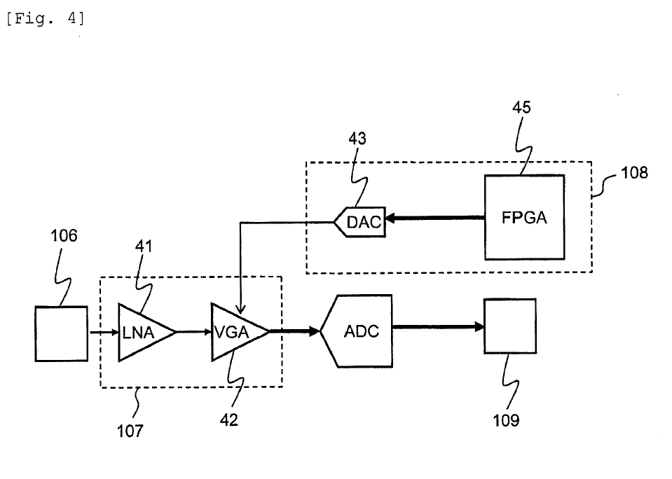

FIG. 3A shows an example of the gain control table which is used when light is irradiated from both sides. The ordinate is gain (dB), and the abscissa is an elapsed time, since light is irradiated. The gain control unit 108 corrects attenuation of the fluence by setting a gain according to the gain control table in the amplifier 107. FIG. 3B shows an example of the gain control table which is used when a light is irradiated from the right side (the same side as the acoustic wave detector 106) in FIG. 1, and FIG. 3C shows an example of the gain control table which is used when light is irradiated from the left side (the opposite side of the acoustic wave detector 106) in FIG. 1.

The fluence distribution inside the subject changes not only by changing position(s) and a number of light irradiation areas (irradiation surface(s)), but also by changing such conditions as the size of the light irradiation area, and the fluence and wavelength of the irradiated light. The fluence distribution inside the subject also changes by changing the thickness of the subject. The arrival time (arrival sequence) of the acoustic wave from each area changes depending on the area of the subject 103 where the acoustic wave detector 106 is contacted. Therefore the gain control tables are preferably prepared in advance for all the possible measurement conditions the subject information acquisition apparatus may require. The measurement conditions can be automatically switched by the subject information acquisition apparatus according to the operation state, measurement purpose or the like, or may be switched by the specification of the user (operator).

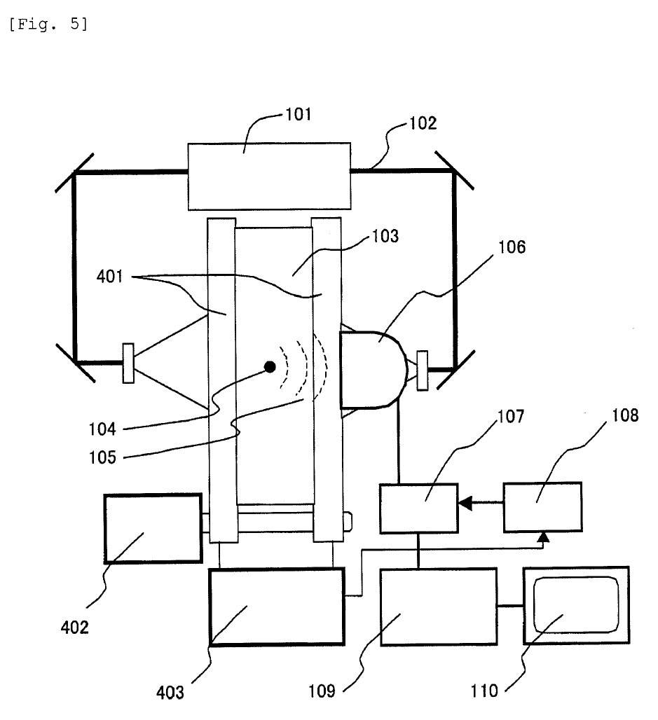

FIG. 4 shows a concrete configuration example of the amplifier 107 and the gain control unit 108 for executing TGC. In FIG. 4, the gain control unit 108 is comprised of a FPGA (Field Programmable Gate Array) 45 and a DA converter (Digital-Analog Converter: DAC) 43. In the memory of the FPGA 45, a plurality of gain control tables are stored. When information on measurement conditions is received from a control system, which is not illustrated, upon measurement, the FPGA 45 reads a gain control table corresponding to the measurement conditions. The FPGA 45 outputs a gain value synchronizing with the acoustic wave detection time. The gain value is converted into analog signals by the DAC 43,and is sent to the amplifier 107. The amplifier 107 is comprised of a low noise amplifier (LNA) 41, and a variable gain amplifier (VGA) 42. The low noise amplifier 41 simply amplifies the signals detected by the acoustic wave detector 106. Then the detected signals are amplified with a gain by the variable gain amplifier 42 according to the detection time. The output of the variable gain amplifier 42 is converted into digital data by an Analog-Digital converter (ADC), and is then sent to the signal processing unit 109.

As described above, according to the configuration of this embodiment, an appropriate TGC can be used for a detected signal of the acoustic wave detector 106 by switching to an appropriate gain control table according to the measurement condition. Therefore a drop in intensity of the acoustic wave caused by attenuation of the fluence can be accurately corrected, and as a result, quality of the reconstructed image, representing the information inside the subject, can be improved.

### (Details of Apparatus)

The configuration of the subject information acquisition apparatus of this embodiment will now be described in detail.

In FIG. 1, the light source 101 is a means of irradiating a light with a specific wavelength which is absorbed by a specific component, out of the components constituting a biological tissue. The light source has at least one pulse light source which can generate pulsed light at a several nano to a several hundred nano second order. The power supply is preferably laser, but a light emitting diode or the like may be used instead of a laser. For the laser, various lasers, including a solid laser, gas laser, dye laser and semiconductor laser, can be used. In this embodiment, an example of using a single light source is shown, but a plurality of light sources may be used. In the case of a plurality of light sources, a plurality of light sources which oscillate a same wavelength may be used in order to increase the irradiation intensity of the light irradiated onto biological tissue, or a plurality of light sources of which oscillation wavelengths are different, may be used in order to measure the difference in the optical property distribution depending on the wavelength. If a dye laser, which can convert an oscillating wavelength, or an OPO (Optical Parametric Oscillator) can be used for the light source, and the difference in the optical property distribution due to wavelength can be measured. The wavelength to be used is preferably 700 nm to 1100 nm, of which absorption is low in biological tissue. To determine the optical property distribution of a biological tissue located relatively near the surface of the biological tissue, a wider range than the above mentioned wavelength region, such as 400 nm to 1600 nm wavelength region, may be used.

The light 102 irradiated from the light source can also be propagated using an optical waveguide. An optical fiber is preferable as the optical wave guide, although this is not illustrated in FIG. 1. In the case of using an optical fiber, a plurality of optical fibers may be used for each light source so as to guide light to the surface of a biological tissue, or lights from a plurality of light sources may be guided to one optical fiber, so as to guide all the lights into a biological tissue using only one optical fiber. The optical apparatus is comprised of such optical components as a mirror which reflects light, and lenses for collecting light, magnifying light or changing light shape, for example. These optical components are arbitrary only if the light 102 emitted from the light source is irradiated onto the light irradiation region on the surface of the subject to be a desired shape.

The subject information acquisition apparatus of this embodiment aims to diagnose malignant tumors, vascular disorders or the like of individuals and animals, and to observe the progress of chemotherapy. Therefore the subjects 103 to be assumed are such diagnostic target areas as a breast, finger and limbs of human and animal bodies. The light absorber is an area in the subject which indicates a high absorption coefficient, and examples are hemoglobin, blood vessels or malignant tumor which includes a high level of hemoglobin, if the measurement target is a human body.

The acoustic wave detector (probe) 106 has one or more element(s) for detecting acoustic waves (ultrasonic waves) generated from an object which absorbed a part of the energy of light propagating in a biological body, converting the acoustic waves into electric signals (detection signals). Any acoustic wave detector can be used if acoustic waves can be detected, such as a transducer using piezoelectric phenomena, a transducer using the resonance of light, and a transducer using a change of capacity. An acoustic wave detector having a plurality of elements may be disposed on the surface of a biological tissue, or an acoustic wave detector having one or more element(s) may scan the surface of a biological tissue two-dimensionally, since the same effect as above can be obtained if acoustic waves can be detected at a plurality of locations. It is preferable to use an acoustic impedance matching agent, such as gel and water, between the acoustic wave detector 106 and the subject, in order to suppress reflection of the sonic waves.

### Second Embodiment

FIG. 5 is a diagram depicting a configuration of a subject information acquisition apparatus according to a second embodiment of the present invention. The subject information acquisition apparatus of this embodiment has a compressing unit for compressing a subject 103, and a standard gain control table is corrected according to the compressing distance, and the corrected table is used for gain control. The rest of the configuration is the same as the first embodiment.

According to the subject information acquisition apparatus of this embodiment, light can be irradiated onto the subject 103 from both sides, in a state of compressing the subject 103 between plate-like members (hereafter called “compressing plates” 401), which face each other. Upon measurement, the compressing distance (distance between the compressing plates 401) is changed by a compressing mechanism 402, so as to deform the subject 103. Thereby the distance from the light irradiation surface to the center portion of the subject 103 is decreased, and fluence that reaches the center portion is increased.

The compressing distance must be determined depending on the size and hardness of the subject 103. If the subject 103 is too large or too hard to compress down to the standard compressing distance, the compressing distance becomes inevitably long. If the subject is smaller than the standard compressing distance, on the other hand, the compressing distance is decreased so that the subject can be interposed.

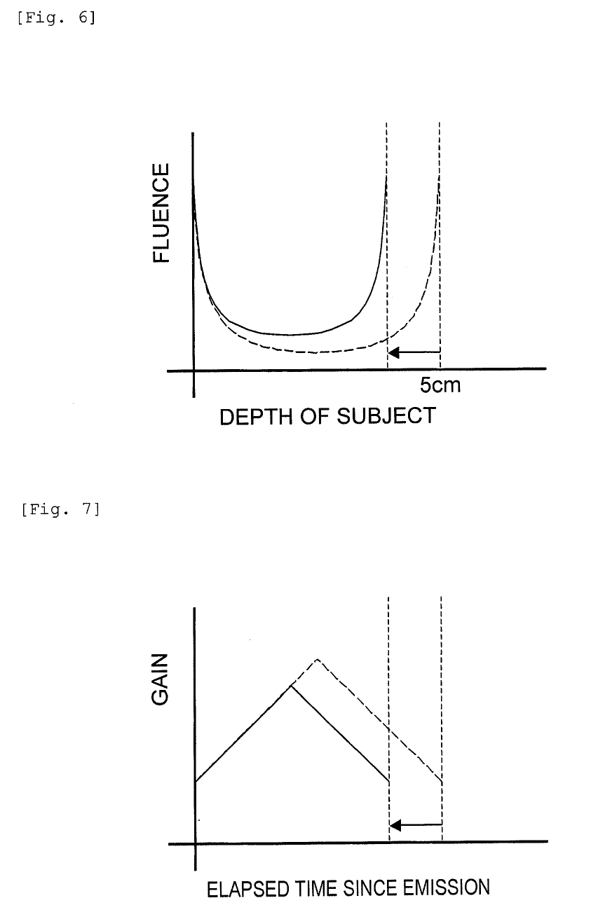

In any case, if the compressing distance changes, the fluence attenuation state inside the subject changes. FIG. 6 is a graph depicting the fluence inside the subject when the compressing distance becomes shorter than a standard compressing distance (e.g. 5 cm). If the compressing distance is decreased, the distance from the light irradiation surfaces on both sides to the inner area of the subject decreases. Since this decreases attenuation of fluence, the fluence inside the subject increases compared with the case of the standard compressing distance. FIG. 7 shows a gain control table in this case, which is generated in the gain control unit 108 for correcting the attenuation of fluence. Since the fluence which reaches the inner area of the subject increases, the gain with respect to the acoustic wave generated in the center portion of the subject can be smaller compared with the standard case. The acoustic wave detection time generally becomes short. Therefore compared with a standard gain control table, the profile of the gain control table is shorter in the time direction, and has a lower peak of the gain at the center portion of the subject. It is preferable to determine the absolute value of the gain considering other factors as well, such as noise of the system.

According to this embodiment, the compressing plates 401 for compressing the subject 103 are driven by the compressing unit 402. The compressing distance is measured by a compressing distance measuring instrument 403 which is the distance measurement unit, and the distance information is input to the gain control unit 108. If the compressing distance is a standard value (5 cm), the gain control unit 108 uses the standard gain control table as is. If the compressing distance is different from the standard value, the gain control unit 108 corrects the gain control table based on the difference of the compressing distances. In concrete terms, if the compressing distance is longer than the standard value, the gain control table is extended in the time direction, and the height of the peak of the gain (maximum value of the gain) is increased, and if the compressing distance is shorter than the standard value, the gain control table is reduced in the time direction, and the height of the peak of the gain (maximum value of the gain) is decreased. Here the position of the peak of the gain (maximum value of the gain) is essentially set to the lowest fluence position inside the subject. To irradiate the subject from both sides with an equal fluence, the peak of the gain is set to the mid-point between the two compressing plates 401. If fluences irradiated from both sides are different, the position of the peak of the gain is determined estimating attenuation of fluences on both sides. In the case of irradiating light onto the subject only from one side as well, the position of the peak of the gain (maximum value of the gain) is essentially set to the lowest fluence position inside the subject.

By performing gain control the same as the first embodiment using a gain control table corrected like this, the intensity of the detected signal can be corrected appropriately. In this embodiment as well, correction accuracy can be further improved if the gain control table is determined considering the attenuation of the acoustic wave itself.

The present invention can also be implemented by executing the following processing. In other words, software (programs) for implementing the functions of each of the above mentioned embodiments is supplied to a system or apparatus via a network or various storage media, and the computer (e.g. CPU, MPU) of the system or apparatus reads and executes the program(s).

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

Aspects of the present invention can also be realized by a computer of a system or apparatus (or devices such as a CPU or MPU) that reads out and executes a program recorded on a memory device to perform the functions of the above-described embodiment(s), and by a method, the steps of which are performed by a computer of a system or apparatus by, for example, reading out and executing a program recorded on a memory device to perform the functions of the above-described embodiment(s). For this purpose, the program is provided to the computer for example via a network or from a recording medium of various types serving as the memory device (e.g., non-transitory computer-readable medium).

1.-2. (canceled)

3. A photoacoustic apparatus comprising:
an acoustic wave detector which detects an acoustic wave which is generated from a subject by irradiating light and outputs a detection signal;
an amplifier which amplifies the detection signal which is output from the acoustic wave detector;
a gain controller which changes a gain of the amplifier as time elapses, according to a predetermined time-based change of the gain such that a gain for a detection signal corresponding to an acoustic wave generated from a source inside the subject receiving a first light fluence is greater than a gain for a detection signal corresponding to an acoustic wave generated from a source inside the subject receiving a second light fluence greater than the first light fluence; and
a signal processor which obtains information inside the subject based on the signal amplified by the amplifier, wherein
the gain controller sets the predetermined time-based change of the gain according to the measurement condition.
